(19) United States
(12) Patent Application Publication
(10) Pub. No.: US 2012/0089202 A1
(43) Pub. Date: Apr. 12, 2012

(54) Flexible Coil Design for Implantable Device

(75) Inventor: Erwin Staller, Innsbruck (AT)

(73) Assignee: MED-EL ELEKTROMEDIZINISCHE GERAETE GMBH, Innsbruck (AT)

(21) Appl. No.: 13/253,313

(22) Filed: Oct. 5, 2011

Related U.S. Application Data
(60) Provisional application No. 61/390,242, filed on Oct. 6, 2010.

(51) Int. Cl. A61N 1/00; H01F 38/14

(52) U.S. Cl. 607/61; 307/104

(57) ABSTRACT

A coupling coil is described for transcutaneous coupling of energy and communications signal in an implantable implant system. The coupling coil has a defined coil plane and multiple concentric curved planar surfaces of conductor and insulation laminate which are arranged perpendicular to the coil plane.

This application claims priority to U.S. Provisional Patent Application 61/390,242, filed Oct. 6, 2010; incorporated herein by reference.

### TECHNICAL FIELD

The present invention relates to a flexible coil design for implantable biomedical devices and systems.

### BACKGROUND ART

Implantable biomedical devices and systems such as cochlear implant systems use inductive and RF Links to transmit energy and/or communications signals over short distances. These arrangements need to be compact, reliable and cheap, but also efficient to allow continuous battery powered operation.

FIG. 1 shows one typical example of a conventional coupling coil for transcutaneous coupling of a communications signal in a cochlear implant system according to the prior art. The conventional coupling coil shown has a defined coil plane and a plurality of concentric coil solid or stranded HF-litz wires that lie in the coil plane. Forms B and C in FIG. 1 show that sometimes, the wires are stacked vertically on each other perpendicular to the coil plane.

Coupling coil design is a major task requiring special know-how and needing a great deal of testing. There is no one simple solution for most coupling coils, but there are design rules and known good reference designs which may be re-used. These coupling coils need a clearly defined geometry which is hard to manufacture in exact sizes. Much effort has gone into the fine-tuning the design of these coupling coils.

### SUMMARY

Embodiments of the present invention are directed to a coupling coil for transcutaneous coupling of an energy and/or communications signal in an implantable biomedical system. The coupling coil has a defined coil plane and multiple concentric curved planar surfaces of conductor (e.g. copper) and insulation laminate which are arranged perpendicular to the coil plane. For example, the curved planar surfaces may be concentric cylinder surfaces or concentric spiral surfaces.

There may also be electronic component package integrated into the coupling coil and containing at least one electronic component in electrical connection with the coupling coil. And there may be at least one coil tap on one of the cylindrical surfaces for electrical connection to the coupling coil. In some embodiments, the coupling coil may form a loose spiral shape. And the conductor and insulation laminate material may include a polymide flexfoil spacer material.

Embodiments of the present invention also include an implantable biomedical system such as a cochlear implant system or other hearing implant system having a coupling coil according to any of the foregoing.

### BRIEF DESCRIPTION OF THE DRAWINGS

FIG. 1 shows an example of a conventional coupling coil according to the prior art.

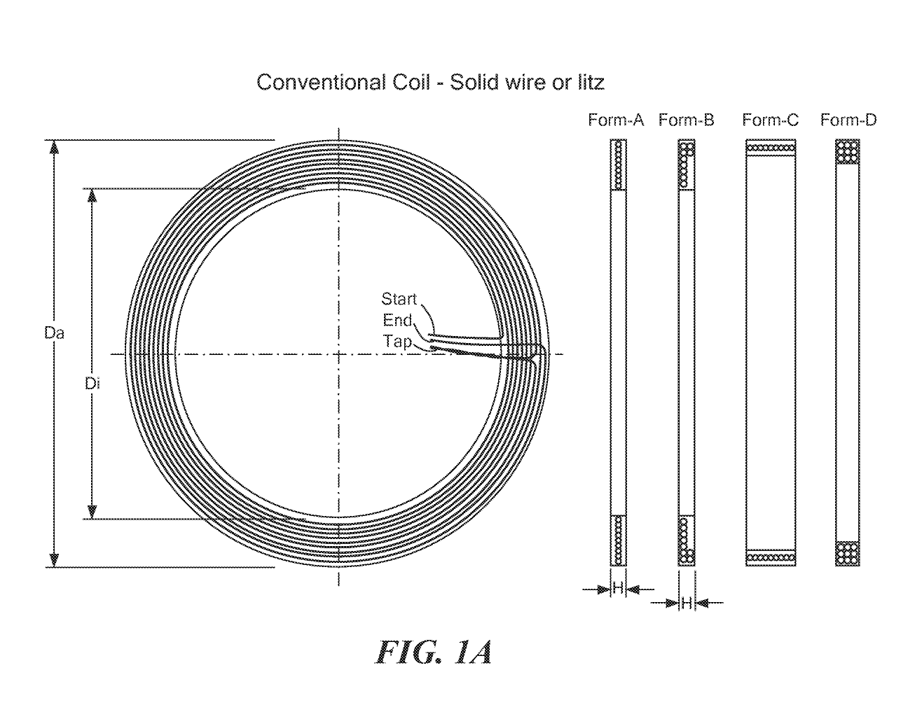

FIG. 2 shows an example of a coupling coil according to one embodiment of the present invention.

FIG. 3 shows a side view of an unrolled laminate surface according to an embodiment of the present invention.

FIG. 4 shows a side view of an unrolled laminate surface according to another embodiment of the present invention including additional electronic components

FIG. 5 shows an example of an embodiment having a single flexible laminate strip wound into a coupling coil with a constant spacer strip.

FIG. 6 shows an example of an embodiment having a single flexible laminate strip wound into a coupling coil with an increasing spacer strip to form a loose spiral.

### DETAILED DESCRIPTION

Various embodiments of the present invention are directed to transcutaneous coupling of a communications signal in an implantable biomedical system. As used herein, the term “communications signal” is given a broad meaning that generally covers electromagnetic energy waves that may or may not rigorously be a signal that contains information, but also broadly includes using electromagnetic energy waves to convey an energy component transcutaneously across the skin of a patient as is useful in implantable biomedical systems such as cochlear implant systems.

In contrast to conventional coupling coils, embodiments of the present invention use a coupling coil having a 90° “vertically flipped” layout approach that allows a tight packing of the coil windings and several electrically independent interleaved coupling coils with taps within one physical coil. Such an approach is a relatively simple design that offers low cost, reliable, easy to manufacture, and provides good performance.

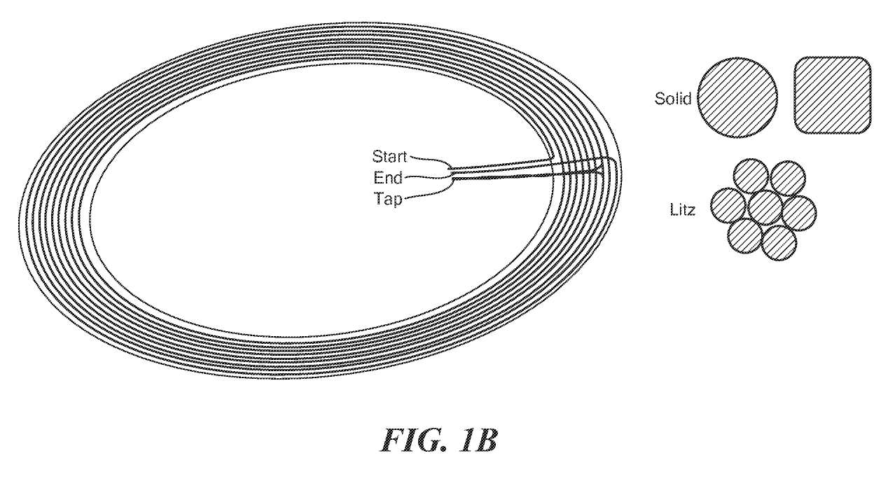

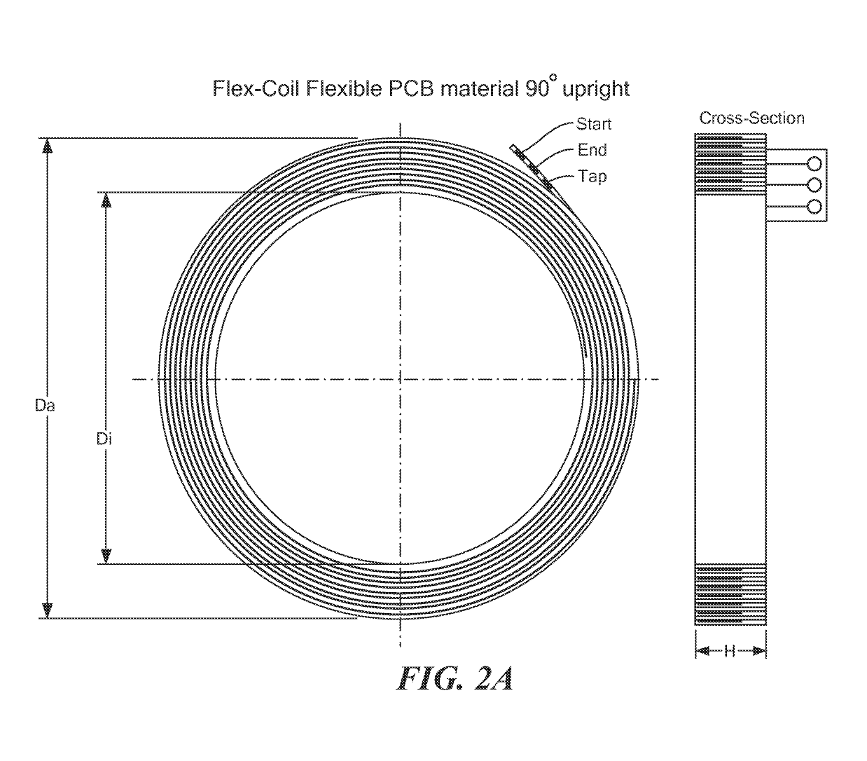

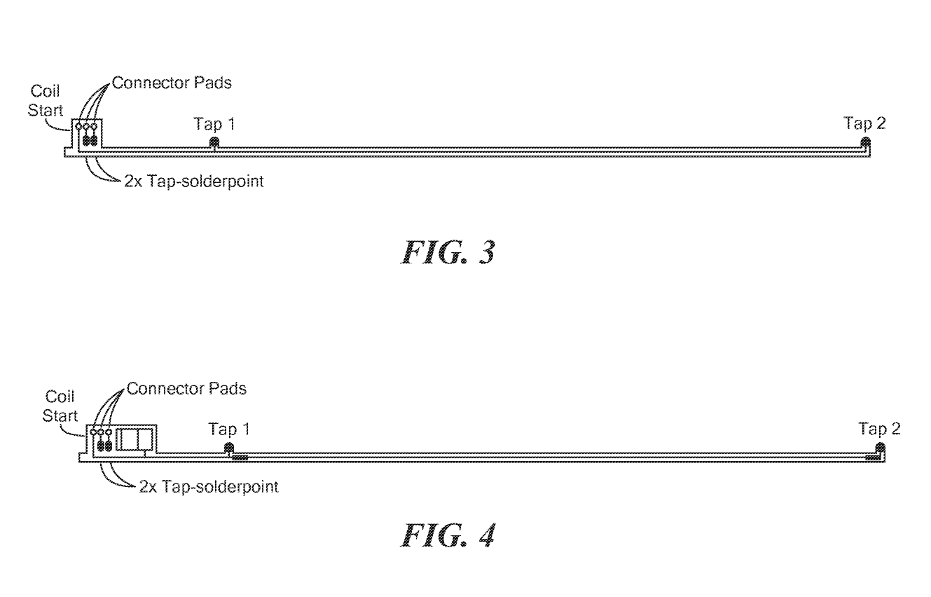

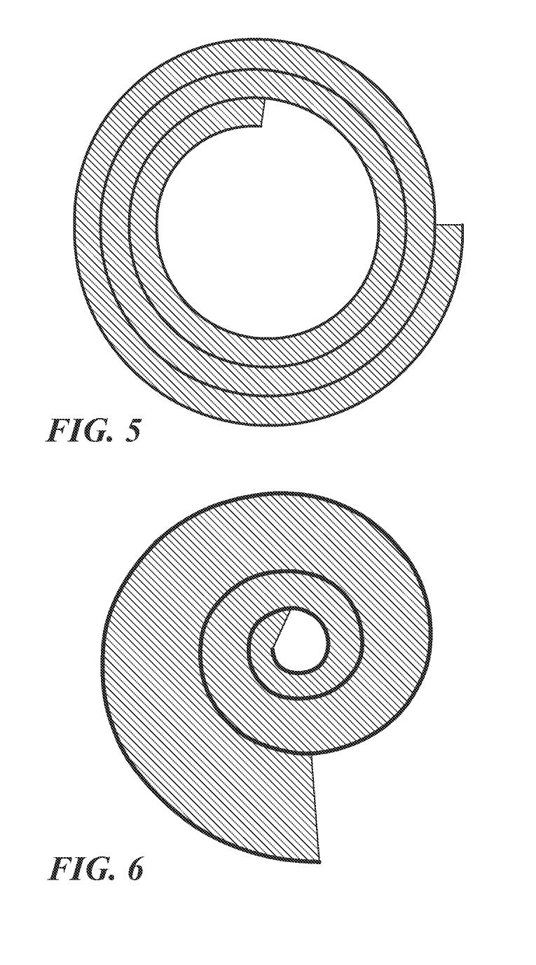

FIG. 2 shows an example of a coupling coil according to one embodiment of the present invention, and FIG. 3 shows a side view of one laminate surface of such an embodiment. The coupling coil has a defined coil plane and multiple concentric curved planar surfaces of conductor (e.g. copper) and insulation laminate which are arranged perpendicular to the coil plane. For example, the curved planar surfaces may be concentric cylinder surfaces as shown in FIG. 5 or concentric spiral surfaces as shown in FIG. 6.

Mass production of such a coupling coil is relatively simple, low cost and easy to scale, enjoying high reproducibility in that the coupling coil is manufactured as a flat, flexible conductor and insulation laminate strip using a manufacturing process of flexible PCBs. Dimensions of the coupling coil are easily reproducible in a tolerance range of 50 μm, and the flexible PCB laminate strip can be easily connected to—or be part of a PCB assembly (e.g., starflex-type boards) carrying other electronic components associated with the biomedical implant system.

The vertical flip approach offers advantageous performance characteristics in a coupling coil for an implantable biomedical system. For example, one important factor in coupling coil design is the consideration of the skin-effect: For frequencies in the range of 10 MHz an intrusion-depth of about 21 μm is typical, and therefore it makes sense to use a conductor which has an optimized surface geometry. In the past, this has mainly been done using specialized HF-Litz stranded wire which has the unfortunate disadvantage of electrical “collapse” seen due parallel capacities of the individual litz strands when the frequency exceeds several MHz. By contrast, the vertical curved planar surfaces of embodiments of the present invention use upright standing conductors that are comparable to 90° twisted conventional printed circuit lines. This allows dramatically increasing the cross-sectional outline of the wire, for example, 2.1 mm of conductive surface outline for a single 1 mm×35 μm PCB-line. Various different diameters are possible for the coupling coil. For cochlear implant applications an exemplary preferable diameter would be around 25 mm. However, diameters around 10 mm or less and 100 m or more are also possible. The choice of the appropriate diameter depends on the targeted application.

The conductor component of the coil is covered by a laminated foil that prevents mechanical and corrosive abrasion of the conductor material substrate. Typically, the conductor and insulation laminate material may be a polymide flexfoil spacer material. After assembly of the coupling coil, the coil windings may be glued together to additionally stabilize the entire structure.

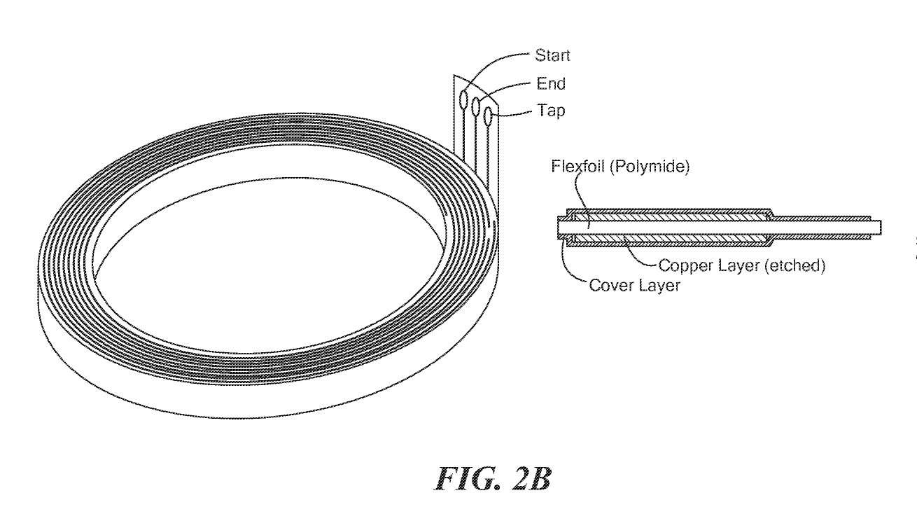

An integration of electronics and coil in one component package becomes possible. As seen in FIG. 3, there may be one or more coil taps on one of the coil surfaces for electrical connection to the coupling coil. FIG. 4 shows a side view of one laminate surface according to another embodiment of the present invention where an electronic component package is integrated into the coupling coil. Depending on the specific device, system and application, the electronic component package may contain one or more electronic components such as switches and/or filters directly in parallel/serial electrical connection with the coupling coil. This can allow a dynamic adaptation of coil parameters such as the number of coil windings, coil inductance and influence on the field geometry of the coupling coil. There may also be several outlets at various positions as denoted by START, END, TAP in FIG. 2. For example, if it turns out during a fitting session that the skin flap of a patient is unexpectedly thick and therefore the transcutaneously transmitted communication signal does not meet the required quality, one or more additional windings of the coupling coil may be added by appropriate switching functions.

The electronic component package may also contain amplification devices, e.g. an amplifier for telemetry signals, in parallel/serial electrical connection with the coupling coil. This can allow dynamic adaptation of the coil signal without having long wires between coil and amplifier. This arrangement may be e.g. advantageous preventing capturing of unwanted HF-signals.

Adaptation of the thickness of the insulator spacer material allows specific control of inter-winding capacitances, and also allows area-covering curves like loosely wound spirals. FIG. 5 shows an example of compact size embodiment having a single flexible laminate strip wound into a coupling coil with a constant spacer strip. FIG. 6 shows an example of an embodiment having a single flexible laminate strip wound into a coupling coil with an increasing spacer strip layer to form a loose spiral. This configuration may support a directional characteristic of the coil.

The wound coupling coil may also have a form more like a square or any other geometric figure in order to fit inside or outside to a housing which may be connected to the coupling coil. Due to its vertical extend and its fairly high mechanical stability the coupling coil may substantially contribute to mechanical robustness of the housing if the coil is placed inside the housing.

Coupling coils as presented in this application may be used for medical implants such as hearing implants, in particular cochlear implants. They may be used without limitation e.g. for the external portion of the implant as in nowadays partially implantable cochlear implants or for recharger modules if the implantable portion contains a rechargeable battery. These coupling coils may also be used for the implantable portion of a partially implantable medical device. Alternatively, these coupling coils may be used for both the implantable and the external portion of a medical device.

Although various exemplary embodiments of the invention have been disclosed, it should be apparent to those skilled in the art that various changes and modifications can be made which will achieve some of the advantages of the invention without departing from the true scope of the invention.

What is claimed is:

1. A coupling coil for a biomedical device comprising:
a coupling coil for transcutaneous coupling of a communications signal in an implantable biomedical system, the coupling coil having a defined coil plane and a plurality of concentric curved planar surfaces of a conductor and insulation laminate perpendicular to the coil plane.

2. A coupling coil according to claim 1, further comprising:
an electronic component package integrated into the coupling coil and containing at least one electronic component in electrical connection with the coupling coil.

3. A coupling coil according to claim 1, further comprising:
at least one coil tap on one of the cylindrical surfaces for electrical connection to the coupling coil.

4. A coupling coil according to claim 1, wherein the coupling coil forms a loose spiral shape.

5. A coupling coil according to claim 1, wherein the conductor and insulation laminate material includes a polymide flexfoil spacer material.

6. A coupling coil according to claim 1, wherein the curved planar surfaces include concentric cylinder surfaces.

7. A coupling coil according to claim 1, wherein the curved planar surfaces include concentric spiral surfaces.

8. An implantable biomedical system having a coupling coil according to any of claims 1-7.

9. A hearing implant system having a coupling coil according to any of claims 1-7.
